(19) United States
(12) Patent Application Publication
(10) Pub. No.: US 2014/0214026 A1
(43) Pub. Date: Jul. 31, 2014

(54) CATHETER

(71) Applicant: BIOTRONIK AG, Buelach (CH)

(72) Inventor: Nicolas Degen, Beringen (CH)

(21) Appl. No.: 14/157,469

(22) Filed: Jan. 16, 2014

Related U.S. Application Data
(60) Provisional application No. 61/757,238, filed on Jan. 28, 2013.

(51) Int. Cl. A61B 18/00

(52) U.S. Cl. CPC A61B 18/00
USPC 606/41

(57) ABSTRACT

A catheter comprising a shaft (1) and a distal tip (3). In order to simplify renal ablation, for example, and to shorten the amount of time the patient is exposed to pain, a torsionally stiff wire (5) rotatable about the axis thereof is disposed between the shaft (1) and the tip (3), the wire being connected to a preloaded film (7, 7.1, 7.2, 7.3, 7.4) rolled up around the wire (5), on the outer surface (8) of which at least one conductor point (9), preferably a plurality of conductor points, is disposed, wherein each conductor point (9) can be connected to a current and/or voltage source.

### CROSS REFERENCE TO RELATED APPLICATIONS

This application claims benefit of priority to U.S. provisional patent application Ser. No. 61/757,238 filed Jan. 28, 2013; the content of which is herein incorporated by reference in its entirety.

### TECHNICAL FIELD

The present invention relates to a catheter comprising a shaft and a distal tip.

### BACKGROUND

Catheters are generally described as tubes or hoses having different diameters and made of different materials, which can be used to probe, drain, fill, rinse or otherwise minimally-invasively treat hollow organs such as the bladder, stomach, intestine, blood vessels or the heart. The tubular or hose-shaped section of the catheter that is introduced into the body of a human or animal to be treated is referred to as the shaft, on the distal end of which the catheter tip is disposed, wherein, of all the catheter elements, the catheter tip penetrates the body furthest.

Intravascular catheters that comprise an active or passive electrode and are used primarily in the cardiac-thoracic region are already known; these catheters are introduced into main veins or arteries, for example into the femoral vein, and, from there, can be advanced to different points in the heart or to the coronary arteries. These catheters are used to depict or stimulate the electrical activity of the heart or to ablate regions having abnormal electrical activity. The latter-mentioned ablation therapy is used to treat cardiac arrhythmias, for example.

Furthermore, catheters are currently used, for example, to lower blood pressure via neuromodulation of the renal nerves (plexus renalis). This therapy is based on the fact that the sympathetic nerve cord adjacent to the renal artery influences blood pressure. In this context it is known that partial traumatization of this nerve results in a sustained reduction of blood pressure and that this treatment is promising, in particular, for patients who are resistant to treatment, that is, for whom conventional pharmacological approaches are not effective or have become ineffective.

Currently, a so-called single-pole system comprising a single electrode for ablation is used for such neuromodulation of the nerve. The physician must reposition the system for every treatment point. Every single ablation on the nerve is painful to the patient, and so the sequential procedure using the single-pole system is often perceived as unpleasant to the patient and the physician. A further disadvantage of this system is that a large portion of the electrode output is not thermally converted in the target tissue but rather is taken up by the blood flowing around the electrode. The power output into the blood can also result in unwanted coagulation of the blood in the region of the ablation site.

Document U.S. Pat. No. 5,239,999 makes known an endocardial mapping and/or ablation system for introduction into a chamber of the heart, in which a catheter shaft comprises a flexible strip, which, in a first state, is wound around a tube in the shape of a helix. A plurality of electrodes is provided on the surface of the strip. The catheter is transformed into a second state by rotating and advancing the tube in the longitudinal direction of the catheter; in said second state, the strip is placed via the outer surface thereof against the tissue of a vessel or hollow body cavity to be treated. The combined rotational and translation motion of the catheter tube causes the helix formed by the strip to expand. The disadvantage of this solution is the combined rotational and translational motion of the tube, which often creates difficulties in the handling of the catheter. In addition, the application forces in the known solution are non-uniformly distributed along the length of the strip, and so the end sections of the strip and, therefore, some of the electrodes in particular do not bear securely against the tissue to be treated.

### SUMMARY

The problem addressed by the present invention is therefore that of creating a catheter that permits simpler ablation of the renal nerves at several points simultaneously. The total handling time is therefore reduced and the patient is exposed to procedural pain for a shorter period of time.

The aforementioned problem is solved by the object set forth in the catheter configurations provided herein. In particular, the catheter according to the invention comprises, between the shaft and the distal tip thereof, a torsionally stiff wire rotatable about the axis thereof, the wire being connected to a preloaded, flat film rolled up around the wire, on the outer surface of which at least one conductor point, preferably a plurality of conductor points, is disposed, wherein each conductor point can be connected to a current and/or voltage source.

The advantage of the solution according to the invention is that a plurality of conductor points (electrodes), in particular, can be arranged on the flat film in a two-dimensional pattern, in a way, thereby permitting ablation to be performed simultaneously or sequentially at several points of the renal nerve. There is no need to change the position or even to carry out a fewer number of repositioning steps. The handling time and the patient's exposure to pain are reduced as a result.

To treat the particular tissue or vessel or any other type of hollow bodily cavity, the rolled-up, flat film, which has been preloaded by being rolled up, is unrolled by rotating the wire about the axis thereof. The film is thereby relieved of tension and then bears uniformly against the wall of the vessel to be treated. As a result, every individual electrically conductive conductor point located on the surface of the film is placed immediately and directly against the inner surface of the vessel or any other type of hollow bodily cavity, thereby ensuring direct electrical contact to the tissue. The film fits snug against the tissue, as it were, since the film attempts to transition into the unrolled, unloaded state, in a manner similar to that of a coiled spring. The conductor points bearing against the tissue are galvanically insulated against the bloodstream to the greatest possible extent, thereby preventing current from flowing into the blood. Therefore, a lower current intensity or less power can be used for sclerosing, thereby potentially reducing the risk of secondary burns to the tissue.

The two-dimensional arrangement of the conductor points on the film in the form of a pattern defines the spatial distribution of the ablation poles. The conductor points can be supplied via the connection to the current and/or voltage source with a high-frequency current for sclerosing the nerves close to the vessel wall. The conductor points are cooled during this procedure by the blood flowing along the back side (inner surface) of the film. Advantageously, the system works without interruption of the physiological blood flow.

In a particularly preferred embodiment of the catheter according to the invention, a sleeve having a slot is also provided, in which said sleeve the wire is disposed and the rolled-up film can be accommodated, wherein the film can be unrolled through the slot. The sleeve protects the film and the conductor points disposed thereon while the catheter is being advanced to the point in the body to be treated. Preferably, the film is disposed in the sleeve in the state with the film rolled up such that the section of the film furthest from the connection point with the wire lies on the outside of the surface of the sleeve and covers said sleeve at least partially or completely. Fastening the film at an outer edge of the slot of the sleeve results in very good clamping behavior of the film.

At least one thermal element and/or one heat-resistant resistor is disposed on the outer surface of the film for performing a local temperature measurement.

In a development of the invention, the film and/or the sleeve is merely slotted in a direction transversely to the longitudinal direction of the catheter and/or is subdivided into at least two sections and equipped with at least one preferably elastic segment, which extends transversely to the longitudinal direction of the catheter and is flexible with respect to bending and/or pressure and/or tension in particular. In the latter variant of this exemplary embodiment, the film and/or the sleeve comprise a plurality of sections, which are connected by the elastic segments. Furthermore, such an elastic segment can also be disposed between the tip (or the shaft) and the film (or the film section) and/or the sleeve (or the sleeve section). The bending resistance of the design can be reduced as a result, thereby enabling the catheter to move laterally.

In a preferred exemplary embodiment of the invention, the film contains a flexible material at least in sections, preferably a polyimide and/or a liquid crystal polymer (LCP).

According to the present invention, the film material functions as carrier material for conductor structures on the outer surface thereof. The conductor points are connected by way of conductor tracks to a corresponding connection disposed on the conductor film, the connection having electrical contact to a voltage and/or current source. An elastic material that undergoes elastic deformation over the long term and exhibits only slight viscoelastic behavior even at temperatures above 50° C. is suitable in particular for the film.

According to the invention, the film preferably has the shape of a rectangle and is sheet-like, wherein, particularly preferably, the film is connected at one lateral edge to the wire. As an alternative, the film, which can optionally comprise multiple layers, can have a different sheet-like shape (e.g. triangular or semicircular), which can vary depending on the intended use (e.g. in accordance with the shape of the organ to be treated). The material of the film is selected such that it is flexurally resilient and any bending is opposed by counter-tension, and so the film is loaded by being rolled up on the wire. The film is relieved of tension when unrolled.

According to the invention, the expression “to roll up” the film means that rotational motion results in a first section of the film being disposed within a further section of the film.

In a further preferred exemplary embodiment, the wire is connected to a cardan drive, which causes the wire to rotate about the axis thereof in order to roll and unroll the film.

In a further exemplary embodiment, the film can comprise a highly thermally conductive material on the inner surface thereof in order to promote cooling of the film, for example by blood or by a coolant (for example, Ringer's solution held at a low temperature) administered externally, or by another bodily fluid. Suitable thermally conductive materials can be noble-metal coatings that are galvanically decoupled from the electrode and also have the required biocompatibility.

The catheter according to the invention having the flexible film and the at least one conductor point on the outer surface thereof can be used as a platform for further vascular, cardiac, visceral or neuronal applications. To this end, any number of sensors and active or passive electrodes can be disposed on the surface of the film. The potential applications are, for example, EKG leads for the esophagus or the trachea, temperature measurements in the esophagus in the case of atrial ablation, neuronal denervations in further organs, stimulations, for example of the baroreceptors in the carotid artery, or measurements of neural activities (chemoreceptors, baroreceptors, MSNA (muscle sympathetic nerve activity)).

Advantageously, when the catheter according to the invention is used in vessels, the fluid (blood or air) contained therein can continue to flow through the particular vessel.

Compared to the prior art according to the document U.S. Pat. No. 5,239,999, in the present invention, the flexible element comprising the electrodes bears against the tissue to be treated in the unloaded state, and therefore all the electrodes lie snug against the tissue in a uniform and direct manner. In addition, according to the present invention, the film is rolled and unrolled using an exclusively rotational motion. Therefore, translatory motion of the catheter is not required in order to place the electrodes against the tissue.

Further objectives, features, advantages, and possible applications of the invention will become apparent from the following description of exemplary embodiments of a catheter according to the invention, with reference to the figures. All of the described and/or graphically depicted features are part of the present invention, either alone or in any combination, independently of their wording in the claims or their back-references.

### DESCRIPTION OF THE DRAWINGS

The drawings show as follows.

FIG. 1 depicts a view of a first exemplary embodiment of a catheter according to the invention from the side in the unloaded state of the film.

FIG. 2 depicts the exemplary embodiment according to FIG. 1 in the loaded state of the film in a cross section, wherein the catheter is disposed in a vessel.

FIG. 3 depicts the exemplary embodiment according to FIG. 1 in the unloaded state of the film in a cross section, wherein the catheter is disposed in a vessel, analogous to FIG. 2.

FIG. 4 depicts a second exemplary embodiment of a catheter according to the invention in a view from the side in the unloaded state.

FIG. 5 depicts the exemplary embodiment according to FIG. 4, also in the unloaded state, wherein the distal section of the catheter is curved.

### DETAILED DESCRIPTION

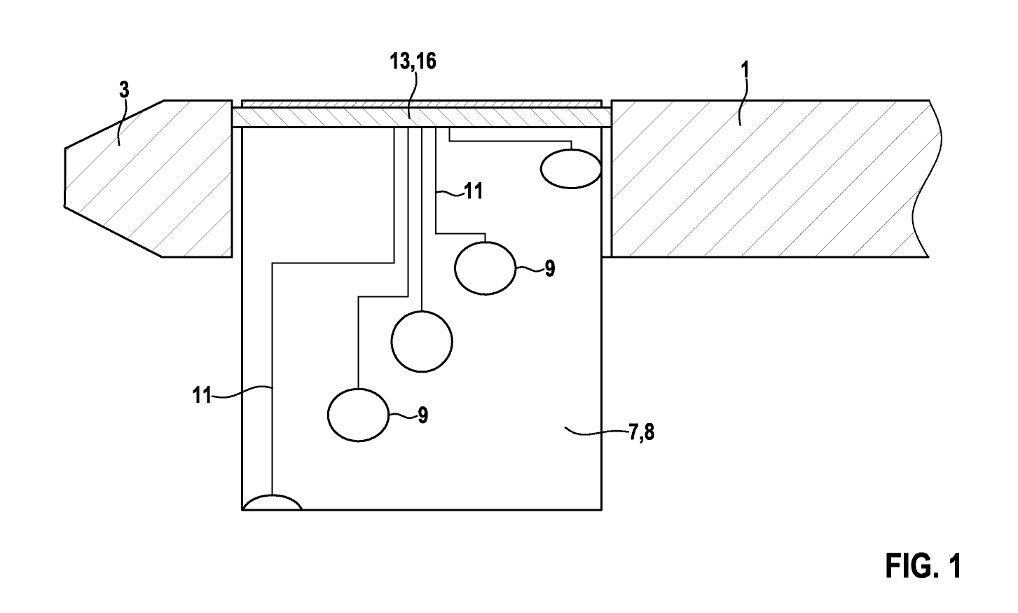

FIG. 1 shows a first exemplary embodiment of a catheter according to the invention comprising a shaft 1, on the distal tip 3 of which a flexible film 7 is disposed, the flexible film being attached to a torsionally stiff wire 5. The elastic film 7 has a substantially rectangular, sheet-type shape, wherein the film 7 is attached via a lateral edge to the wire 5. A plurality of conductor points 9 is disposed on the outer surface (outer side) 8 of the film 7. Each conductor point 9 is connected via a conductor track 11 to a current or voltage source (not shown), which is preferably disposed outside of the catheter on the proximal end thereof. In the first exemplary embodiment of a catheter according to the invention, the conductor points 9 are intended for use, in particular, to sclerose regions of the renal nerves using an appropriate electric current, and therefore each conductor point 9 can be considered an active electrode.

In the first exemplary embodiment, which is shown in FIG. 1, the conductor points 9 are distributed along a diagonal line over the outer side 8 of the film 7. Any other type of arrangement of the conductor points 9 is also feasible. In a further exemplary embodiment, at least one thermal element, at least one sensor and/or at least one passive electrode can also be disposed on the surface of the film 7.

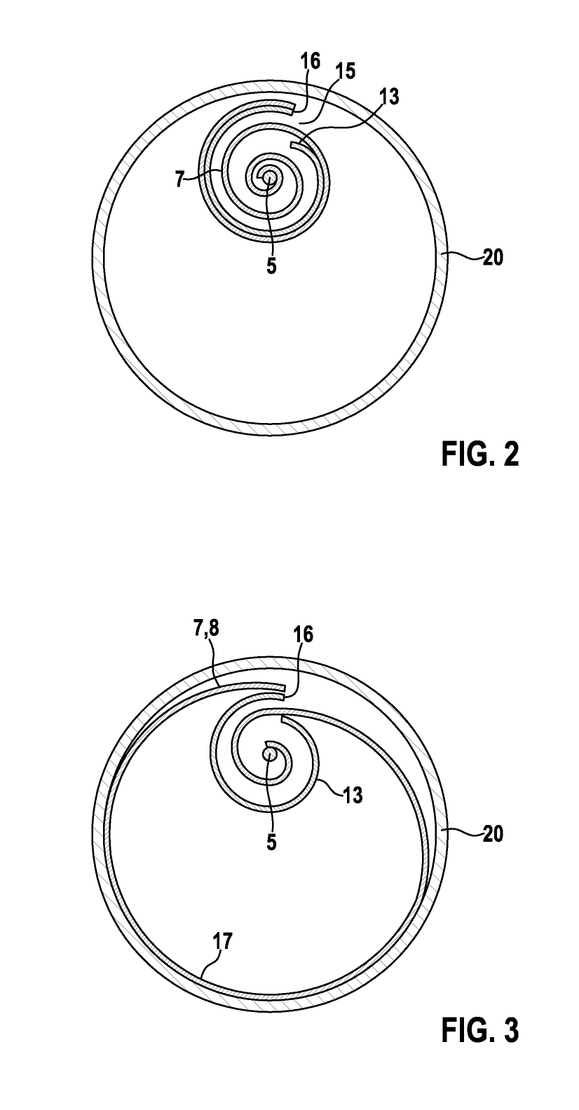

In the loaded state of the film 7 shown in FIG. 2, in which the catheter comprising the film 7 has a small outer diameter and in which the catheter is guided to the point in the patient's body to be treated, the flexible film 7 is disposed such that this is rolled up around the wire 5 in a tubular sleeve 13, wherein the section of the film 7 opposite the wire 5 is placed around the sleeve 13. The film 7 is attached at a lateral edge to the outer edge 16 of the slot 15 of the sleeve 13. The film 7, in combination with the sleeve 13, has the shape and mode of operation of a balance wheel, as it were, which results in very good clamping behavior of the film 7.

In order to perform treatment, for example to perform renal ablation of the renal nerves, the wire 5 is rotated about the longitudinal axis thereof, for example using a cardan drive disposed on the proximal end of the catheter, in such a way that the film 7 is unrolled through a slot 15 of the sleeve 13 that extends parallel to the longitudinal axis of the catheter or the wire 5, and is transformed into the unloaded state, in which the film 7 bears against the inner wall of the vessel 20. In the unloaded state, the section of the catheter in which the film 7 is disposed has a diameter that is markedly greater compared to the loaded state. By way of the transformation into the unloaded state, the conductor points 9 bear directly against the vessel wall, and the spatial distribution of the conductor points 9 allows treatment to be performed at several points on the renal nerves simultaneously or sequentially without further positioning. To this end, the conductor points 9 are supplied via the conductor tracks 11 with a high-frequency current, which scleroses the nerves close to the vessel wall. The blood flowing through the vessel flows past the back side (inner side) 17 of the film 7 and cools said film. Thermal sensors (not shown), which can be disposed in the vicinity of the conductor points 9 on the outer side 8 of the film 7, monitor the temperature and ensure that the maximum permissible temperature in the blood is not exceeded. Therefore, the system can function in a minimally invasive manner without interrupting the physiological blood flow.

In order to improve the heat transfer into the blood, the film 7 can be provided on the inner side 17 with a highly thermally conductive material, for example a noble-metal coating.

Since the snug placement of the conductor points 9 against the wall of the vessel 20 results in insulation against blood, leakage current does not occur. Therefore, less power is required for ablation compared to the known method (a single-pole system, i.e. a counterelectrode placed on the body surface) due, according to the invention, to a nearly complete, heat-resistant conversion of the electrically applied power at or near the target tissue to be therapeutically treated. The more highly targeted and more efficient application of energy into the tissue shortens the treatment time and, therefore, the amount of time the patient is exposed to pain.

Furthermore, the targeted and, therefore, better controlled application of energy minimizes the risk of burns occurring on the vessel wall.

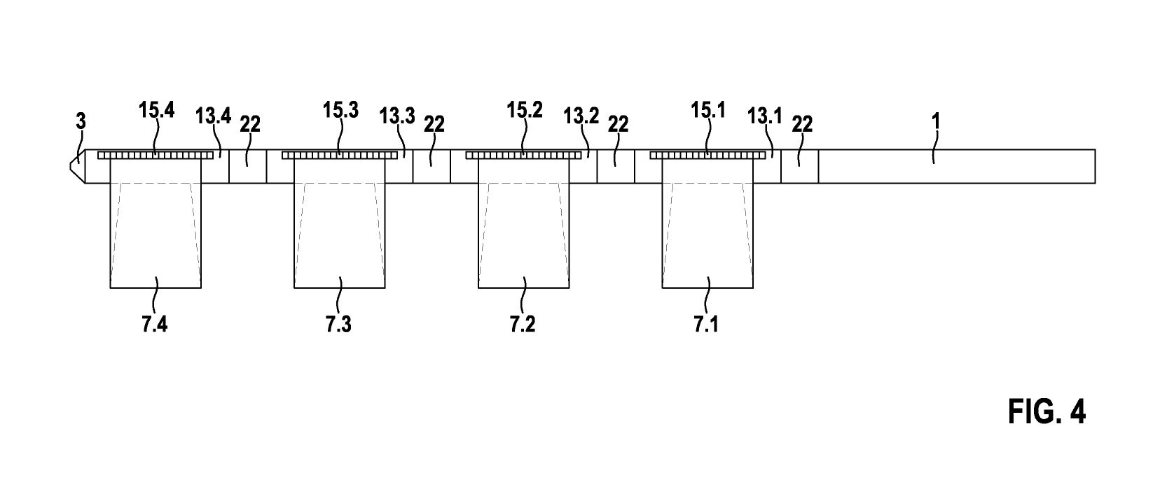

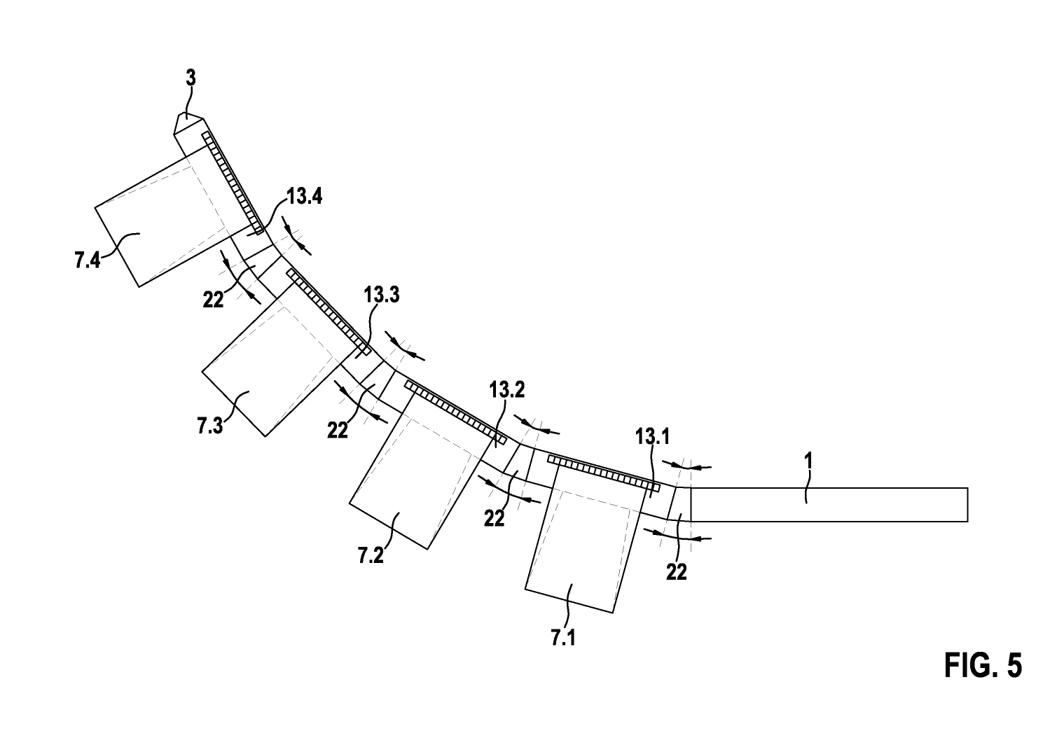

In order to improve the guidance of the catheter according to the invention through the patient's body to the renal nerves, in the second exemplary embodiment, which is shown in FIGS. 4 and 5, the sleeve 13 and the film 7 are subdivided into a total of four film sections 7.1, 7.2, 7.3 and 7.4 and four sleeve sections 13.1, 13.2, 13.3 and 13.4 (each of which comprises a slot 15.1, 15.2, 15.3, 15.4), which are disposed behind one another along the longitudinal direction of the catheter (catheter axis). A flexible, elastic segment 22 is provided between the shaft 1 and the first sleeve and film sections 7.1, 13.1 and between the adjacent film and sleeve sections, said segment connecting the shaft 1 and the sleeve and film sections or two adjacent sleeve and film sections to one another in a manner that is flexible with respect to bending in particular. As a result, as shown in FIG. 5, the catheter according to the invention can be bent and, therefore, guided through the curved sections in the patient's body. The sleeve and film sections 7.1, 7.2, 7.3, 7.4 and 13.1, 13.2, 13.3, 13.4, respectively, are relatively rigid by comparison.

Every sleeve and film section 7.1, 7.2, 7.3, 7.4 and 13.1, 13.2, 13.3, 13.4, respectively, shown in FIGS. 4 and 5 corresponds with respect to design to the sleeve 13 comprising the film 7, including the slot 15.1, 15.2, 15.3, 15.4, the wire 5 and the conductor structure (conductor points 9, conductor tracks 11), wherein the conductor points 9 can be arranged in a different two-dimensional pattern, of course.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

### LIST OF REFERENCE CHARACTERS

1 shaft
3 tip, distal end of the catheter
5 wire
7, 7.1, 7.2, 7.3, 7.4 film
8 outer side of the film 7
9 conductor point
11 conductor track
13, 13.1, 13.2, 13.3, 13.4 sleeve
15, 15.1, 15.2, 15.3, 15.4 slot
16 outer edge of the slot 15
17 inner side of the film 7
20 vessel
22 flexible segment

What is claimed is:

1. A catheter, between the shaft and distal tip of which a torsionally stiff wire rotatable about the axis thereof is disposed, the wire being connected to a preloaded film rolled up around the wire, on the outer surface of which at least one conductor point, optionally a plurality of conductor points, is disposed, wherein each conductor point can be connected to a current and/or voltage source.

2. The catheter according to claim 1, further comprising a sleeve having a slot, in which the wire is disposed and in which the rolled-up film can be accommodated, wherein the film can be unrolled through the slot.

3. The catheter according to claim 1, characterized in that at least one thermal element and/or one heat-resistant resistor is disposed on the outer surface of the film.

4. The catheter according to claim 1, characterized in that the film and/or the sleeve is slotted in a direction transverse to the longitudinal direction of the catheter and/or is subdivided into at least two sections and is equipped with at least one flexible segment, which extends transversely to the longitudinal direction of the catheter.

5. The catheter according to claim 1, characterized in that the film contains a flexible material, at least in sections, optionally a polyimide and/or a liquid crystal polymer (LCP).

6. The catheter according to claim 1, characterized in that the wire is connected to a cardan drive.
